(19) United States
(12) Patent Application Publication
(10) Pub. No.: US 2010/0312030 A1
(43) Pub. Date: Dec. 9, 2010

(54) PROCESS OF SYNTHESIS GAS CONVERSION TO LIQUID FUELS USING SYNTHESIS GAS CONVERSION CATALYST AND NOBLE METAL-PROMOTED ACIDIC ZEOLITE HYDROCRACKING-HYDROISOMERIZATION CATALYST

(75) Inventors: Charles L. Kibby, Benicia, CA (US); Kandaswamy Jothimurugesan, Hercules, CA (US); Tapan K. Das, Albany, CA (US)

Correspondence Address: CHEVRON CORPORATION, P.O. BOX 6006, SAN RAMON, CA, 94583-0806, US

(73) Assignee: Chevron U.S.A., Inc.

(21) Appl. No.: 12/478,204

(22) Filed: Jun. 4, 2009

(51) Int. Cl. C07C 1/04

(52) U.S. Cl. 585/310

(57) ABSTRACT

A process is disclosed for converting a feed comprising synthesis gas to liquid hydrocarbons within a single reactor at essentially common reaction conditions. The synthesis gas contacts a first catalyst bed comprising a synthesis gas conversion catalyst, and a second catalyst bed comprising a mixture of a hydrogenation catalyst and a solid acid catalyst. A Fischer-Tropsch wax is formed over the first catalyst bed and the wax is then hydrocracked and hydroisomerized over the second catalyst bed, resulting in liquid hydrocarbons substantially free of solid wax.

### BACKGROUND OF THE INVENTION

1. Field of the Invention

The invention relates to an improved process for converting synthesis gas to liquid hydrocarbon mixtures useful as distillate fuel and/or lube base oil by contacting the gas with multiple catalysts in a stacked bed arrangement within a single reactor.

2. Description of Related Art

The majority of combustible liquid fuel used in the world today is derived from crude oil. However, there are several limitations to using crude oil as a fuel source. For example, crude oil is in limited supply.

Alternative sources for developing combustible liquid fuel are desirable. An abundant resource is natural gas. The conversion of natural gas to combustible liquid fuel typically involves a first step of converting the natural gas, which is mostly methane, to synthesis gas, or syngas, which is a mixture of carbon monoxide and hydrogen. Fischer-Tropsch synthesis is a known means for converting syngas to higher molecular weight hydrocarbon products. Fischer-Tropsch diesel has a very high cetane number and is effective in blends with conventional diesel to reduce NOₓ and particulates from diesel engines, allowing them to meet stricter emission standards.

Fischer-Tropsch synthesis is often performed under conditions which produce a large quantity of C₂₁+ wax, also referred to herein as “Fischer-Tropsch wax,” which must be hydroprocessed to provide distillate fuels. Often, the wax is hydrocracked to reduce the chain length, and then hydrotreated to reduce oxygenates and olefins to paraffins. Hydrocracking tends to reduce the chain length of all of the hydrocarbons in the feed. When the feed includes hydrocarbons that are already in a desired range, for example, the distillate fuel range, hydrocracking of these hydrocarbons is undesirable.

Considerably different process conditions are required for hydrocracking and hydroisomerization of Fischer-Tropsch wax using relatively acidic catalysts such as ZSM-5 than for Fischer-Tropsch synthesis. For this reason commercial Fischer-Tropsch plants require separate reactors for the Fischer-Tropsch synthesis and for the subsequent hydrocracking of the product wax, and complicated and expensive separation schemes may be required to separate solid wax from lighter products.

For example, U.S. Pat. No. 4,617,288 describes a process whereby synthesis gas is converted to hydrocarbons by flowing the gas first over iron-containing Fischer-Tropsch catalyst and then over a zeolite. The effluent from a first stage reactor is passed directly to a second stage zeolite catalyst conversion reactor. Conditions vary considerably between the two reactors; operating conditions in the first stage are conducted at a temperature between 232° C. to 288° C. while the operating temperature in the second reactor is specified as between 260° C. and 482° C.

Zhao, et al., Ind. Eng. Chem. Res. 2005, 44, 769-775 discloses a process for the synthesis of middle isoparaffins via a two-stage Fischer-Tropsch reaction. In a first catalyst reactor is placed a Fischer-Tropsch synthesis catalyst comprised of mixed particles of Co/SiO₂ and H-ZSM-5 while a second reactor contains a hydrocracking catalyst containing Pd/SiO₂ and H-ZSM-5. It is necessary to operate the second reactor at a temperature 50° C. higher than the first reactor with a further addition of hydrogen in order to obtain reasonable hydrocracking and hydroisomerization rates.

Nam et al., Catalysis Letters, 2009 (on-line early edition) discloses a process for the production of a middle distillate using a dual-bed reactor. In the first bed reactor the Fischer-Tropsch synthesis is conducted under conditions of 220° C., 12 bar and H₂/CO ratio of 2.0, while in the second bed reactor it is necessary to conduct the hydrocracking and hydroisomerization reactions under the more severe conditions of 330° C., 12 bar and an increased hydrogen concentration (H₂/CO of 2.5). The Fischer-Tropsch catalyst employed in the first bed is Co/TiO₂, while in the second bed reactor a catalyst composed of palladium incorporated into a mesoporous acidic alumina is used.

Liu et al., Ind. Eng. Chem. Res., 20065 44, 7429-7336 describes a process for the direct production of gasoline-range isoparaffins from Fischer-Tropsch synthesis using a single reactor bed. The catalyst system consists of a physical mixture of separate particles of Co/SiO₂ and palladium impregnated on zeolite beta. The process avoids the formation of wax as the zeolite interrupts the oligomerization process. Although this process is effective for the conversion of syngas to light gasoline-range hydrocarbon products, the catalyst exhibits fairly rapid deactivation.

It would be advantageous to provide a process in which both synthesis gas conversion and product hydrocracking and hydroisomerization are combined within a single reactor at a common set of conditions.

### SUMMARY OF THE INVENTION

The invention relates to a process for converting synthesis gas to liquid hydrocarbons in a single reactor comprising contacting a feed comprising a mixture of carbon monoxide and hydrogen with a first catalyst bed comprising a synthesis gas conversion catalyst and a second catalyst bed comprising a mixture of a hydrogenation catalyst and a solid acid catalyst downstream of the first bed at an essentially common temperature and pressure, such that a Fischer-Tropsch wax is formed over the first bed and said wax is hydrocracked and hydroisomerized over the second catalyst bed, thereby resulting in liquid hydrocarbons substantially free of solid wax.

### DETAILED DESCRIPTION OF THE INVENTION

A process is disclosed for the synthesis of liquid hydrocarbons in the distillate fuel and/or lube base oil range from synthesis gas in a single fixed bed reactor. Within a fixed bed reactor, multiple, small-diameter tubes are enclosed in a common cooling medium. Provided within the process is a method for synthesizing a mixture of olefinic and paraffinic hydrocarbons by contacting the synthesis gas with a synthesis gas conversion catalyst in a first, upstream catalyst bed. The hydrocarbon mixture so formed can range from methane to light wax, and may include linear, branched and cyclic compounds. The hydrocarbon mixture is then contacted within the same reactor downstream of the first catalyst bed with a mixture of catalysts within a second, downstream catalyst bed. The mixture includes a hydrogenation catalyst for hydrogenating olefins and a solid acid catalyst for hydrocracking and hydroisomerizing the straight chain hydrocarbons. The upstream bed functions as a synthesis gas conversion catalyst while the downstream bed functions as a hydrocracking and hydroisomerization catalyst. Both the synthesis gas conversion and the subsequent hydrocracking and hydroisomerization are carried out in a single reactor under essentially common reaction conditions without having to provide a separate reactor for hydrocracking and hydroisomerization. By “essentially common reaction conditions” is meant that the temperature of the cooling medium within the reactor is constant from one point to another within a few degrees Celsius (e.g., 0-3° C.) and the pressure within the reactor is allowed to equilibrate between the two beds. The temperatures and pressures of the upstream and downstream beds can differ somewhat, although advantageously it is not necessary to separately control the temperature and pressure of the two beds. The bed temperatures will depend on the relative exotherms of the reactions proceeding within them. Exotherms generated by synthesis gas conversion are greater than those generated by hydrocracking, so the average upstream bed temperature will generally be higher than the average downstream bed temperature. The temperature difference between the beds will depend on various reactor design factors, including, but not limited to, the temperature of the cooling medium, the diameter of the tubes in the reactor, the rate of gas flow through the reactor, and so forth. For adequate thermal control, the temperatures of the two beds are preferably maintained within about 10° C. of the cooling medium temperature, and therefore the difference in temperature between the upstream and downstream beds is preferably less than about 20° C., even less than about 10° C. The pressure at the end of the upstream bed is equal to the pressure at the beginning of the downstream bed since the two beds are open to one another. Note that there will be a pressure drop from the top of the upstream bed to the bottom of the downstream bed because gas is being forced through narrow tubes within the reactor. The pressure drop across the reactor could be as high as about 50 psi (3 atm), therefore the average difference in pressure between the beds could be up to about 25 psi.

The upstream and downstream catalyst beds are arranged in series, in a stacked bed configuration.

A feed of synthesis gas is introduced to the reactor via an inlet. The ratio of hydrogen to carbon monoxide of the feed gas is generally high enough that productivity and carbon utilization are not negatively impacted without the addition of hydrogen into the reactor or production of additional hydrogen using water-as shift. The ratio of hydrogen to carbon monoxide of the feed gas is also generally below a level at which excessive methane would be produced. Advantageously, the ratio of hydrogen to carbon monoxide is between about 1.0 and about 2.2, even between about 1.5 and about 2.2. If desired, pure synthesis gas can be employed or, alternatively, an inert diluent, such as nitrogen, CO₂, methane, steam or the like can be added. The phrase “inert diluent” indicates that the diluent is non-reactive under the reaction conditions or is a normal reaction product.

The feed gas initially contacts a synthesis gas conversion catalyst in the upstream bed of the reactor. According to one embodiment, the synthesis gas conversion catalyst can be any known Fischer-Tropsch synthesis catalyst, Fischer-Tropsch catalysts are typically based on group VIII metals such as, for example, iron, cobalt, nickel and ruthenium. Catalysts having low water gas shift activity and suitable for lower temperature reactions, such as cobalt, are preferred. The synthesis gas conversion catalyst can be supported on any suitable support, such as solid oxides, including but not limited to alumina, silica or titania.

According to an alternative embodiment, the upstream bed can use a hybrid synthesis gas conversion catalyst containing a synthesis gas conversion catalyst in combination with an olefin isomerization catalyst, for example a relatively acidic zeolite, for isomerizing double bonds in C₄⁺ olefins as they are formed. Methods for preparing a hybrid catalyst of this type are described in co-pending U.S. patent application Ser. No. 12/343,534, incorporated by reference. Such a method comprises impregnating a zeolite extrudate using a solution comprising a cobalt salt to provide an impregnated zeolite extrudate and activating the impregnated zeolite extrudate by a reduction-oxidation-reduction cycle. Impregnation of a zeolite using a substantially non-aqueous cobalt solution followed by activation by a reduction-oxidation-reduction cycle reduces cobalt ion-exchange with zeolite acid sites, thereby increasing the overall activity of the zeolite component. The resulting zeolite supported cobalt catalyst comprises cobalt metal distributed as small crystallites upon the zeolite support. The cobalt content of the zeolite supported cobalt catalyst can depend on the alumina content of the zeolite support. For example, for an alumina content of about 20 to about 99 weight % based upon support weight, the catalyst can contain, for example, from about 1 to about 20 weight % cobalt, preferably 5 to about 15 weight % cobalt, based on total catalyst weight, at the lowest alumina content. At the highest alumina content the catalyst can contain, for example, from about 5 to about 30 weight % cobalt, preferably from about 10 to about 25 weight % cobalt, based on total catalyst weight. The reduction-oxidation-reduction cycle used to activate the catalyst includes a first reduction step at a temperature in a range of about 200° C. to about 450° C., an oxidation step at a temperature in a range of about 250° C. to about 350° C., and a second reduction step at a temperature in a range of about 200° to about 450° C.

The downstream catalyst bed contains a catalyst mixture including a hydrogenation catalyst for hydrogenating olefins and a solid acid catalyst for hydrocracking and hydroisomerizing the straight chain hydrocarbons. As is well known, hydrocracking catalysts contain a hydrogenation component and a cracking component. The hydrogenation component is typically a metal or combination of metals selected from Group VIII noble and non-noble metals and Group VIB metals. Preferred noble metals include platinum, palladium, rhodium and iridium. Non-noble metals which can be used include molybdenum, tungsten, nickel, cobalt, etc. Where non-noble metals are used It is generally preferred to use a combination of metals, typically at least one Group VIII metal and one Group VIB metal, e.g., nickel-molybdenum, cobalt-molybdenum, nickel-tungsten, and cobalt-tungsten. The non-noble metal hydrogenation metals are usually present in the final catalyst composition as oxides, or more preferably, as sulfides when such compounds are readily formed from the particular metal involved. Preferred non-noble metal overall catalyst compositions contain in excess of about 5 weight percent, preferably about 5 to about 40 weight percent molybdenum and/or tungsten, and at least about 0.5, and generally about 1 to about 15 weight percent of nickel and/or cobalt determined as the corresponding oxides. The sulfide form of these metals is most preferred due to higher activity, selectivity and activity retention.

The hydrogenation component can be incorporated into the overall catalyst composition by any one of numerous procedures. It can be added either to the cracking component, to the support or a combination of both. In the alternative, the Group VIII components can be added to the cracking component or matrix component by co-mulling, impregnation, or ion exchange and the Group VI components, i.e.; molybdenum and tungsten can be combined with the refractory oxide by impregnation, co-mulling or co-precipitation. These components are usually added as a metal salt which can be thermally converted to the corresponding oxide in an oxidizing atmosphere or reduced to the metal with hydrogen or other reducing agent.

The cracking component is an acid catalyst material and can be a material such as amorphous silica-alumina or tungstated zirconia or a zeolitic or non-zeolitic crystalline medium pore molecular sieve. Examples of suitable hydrocracking molecular sieves include zeolite Y, zeolite X and the so called ultra stable zeolite Y and high structural silica:alumina ratio zeolite Y such as for example described in U.S. Pat. Nos. 4,401,556, 4,820,402 and 5,059,567, herein incorporated by reference. Small crystal size zeolite Y, such as described in U.S. Pat. No. 5,073,530, herein incorporated by reference, can also be used. Other zeolites which show utility as cracking catalysts include those designated as SSZ-13, SSZ-33, SSZ-46, SSZ-53, SSZ-55, SSZ-57, SSZ-58, SSZ-59, SSZ-64, ZSM-5, ZSM-11, ZSM-12, ZSM-23, H-Y, beta, mordenite, SSZ-74, ZSM-48, TON type zeolites, ferrierite, SSZ-60 and SSZ-70. Non-zeolitic molecular sieves which can be used include, for example silicoaluminophosphates (SAPO), ferroaluminophosphate, titanium aluminophosphate and the various ELAPO molecular sieves described in U.S. Pat. No. 4,913,799 and the references cited therein. Details regarding the preparation of various non-zeolite molecular sieves can be found in U.S. Pat. No. 5,114,563 (SAPO); U.S. Pat. No. 4,913,799 and the various references cited in U.S. Pat. No. 4,913,799, hereby incorporated by reference in their entirety. Mesoporous molecular sieves can also be included, for example the M41S family of materials (J. Am. Chem. Soc. 1992, 114, 10834-10843), MCM-41 (U.S. Pat. Nos. 5,246,689, 5,198,203, 5,334,368), and MCM48 (Kresge et al., Nature 359 (1992) 710).

The amount of catalyst mixture in the downstream bed can be suitably varied to obtain the desired product. If the catalyst mixture amount is too low, there will be insufficient cracking to remove all of the wax; whereas if there is too much catalyst mixture in the downstream bed, there will be too much cracking and the resulting product may be too light. The amount of catalyst mixture needed in the downstream bed will in part depend on the tendency of the synthesis gas conversion catalyst in the upstream bed to produce wax. In general, the weight of the catalyst mixture in the downstream bed is between about 0.5 and about 2.5 of the weight of the catalyst in the upstream bed. The reaction temperature is suitably from about 160° C. to about 260° C. for example, from about 175° C. to about 250° C. or from about 185° C. to about 235° C. Higher reaction temperatures favor lighter products. The total pressure is, for example, from about 1 to about 100 atmospheres, for example, from about 3 to about 35 atmospheres or from about 5 to about 20 atmospheres. Higher reaction pressures favor heavier products. The gaseous hourly space velocity based upon the total amount of feed is less than 20,000 volumes of gas per volume of catalyst per hour, for example, from about 100 to about 5000 v/v/hour or from about 1000 to about 2500 v/v/hour.

Fixed bed reactor systems have been developed for carrying out the Fischer-Tropsch reaction. Such reactors are suitable for use in the present process. For example, suitable Fischer-Tropsch reactor systems include multi-tubular fixed bed reactors the tubes of which are loaded with the upstream and downstream catalyst beds.

The present process provides for a high yield of paraffinic hydrocarbons in the middle distillate and/or light base-oil range under essentially the same reaction conditions as the synthesis gas conversion. The hydrocarbons produced are liquid at about 0° C. and substantially free of solid wax By “substantially free of solid wax” is meant that the product is a single liquid phase at ambient conditions without the presence of an insoluble solid wax phase. In particular, the process provides a product having the following composition;
0-20, for example, 5-15 or 8-12, weight % CH₄;
0-20, for example, 5-15 or 8-12, weight % C₂-C₄;
50-95, for example, 60-90 or 75-80, weight % C₅₊; and
0-8 weight % C₂₁₊.

In addition, the present process provides for a high yield of paraffinic hydrocarbons in the middle distillate and/or light base-oil range without the need for separation of products arising from the first catalyst bed and without the need for a second reactor containing catalyst for hydrocracking and hydroisomerization. It has been found that with a proper combination of catalyst composition, catalyst bed placement and reaction conditions, both the synthesis gas conversion reaction and the subsequent hydrocracking/hydroisomerization reactions can be conducted within a single reactor under essentially common process conditions.

An additional advantage to the present process is that undesired methane selectivity is kept low as a result of maintaining the process temperature in the lower end of the optimum range for Fischer-Tropsch synthesis and considerably lower than what is generally believed required for adequate hydrocracking and hydroisomerization activity. It is well known that high methane selectivity is found at the elevated temperatures commonly used for hydrocracking and hydroisomerization.

### EXAMPLES

### Example 1

### Preparation of Synthesis Gas Conversion Catalyst Comprising 10 Weight % Co-0.25 Weight % Ru Supported on 72 Weight % ZSM-5 and 20 Weight % Alumina

A catalyst of CoRu (10 weight % Co, 0.25 weight % Ru) on ZSM-5 extrudates was prepared by impregnation In a single step. First, ruthenium nitrosyl nitrate was dissolved in water. Second, cobalt nitrate was dissolved in acetone. The volume ratio of the two solutions was similar to the weight ratios of the metals (i.e., 40 acetone:1 water). The two solutions were mixed together and then added to 1/16″ extrudates of alumina (20 weight % alumina) bound ZSM-5 zeolite (Zeolyst CBV 014 available from Zeolyst International, having a Si/Al ratio of 40). After the mixture was stirred for 1 hour at ambient temperature, the solvent was eliminated by rotavaporation, also at ambient temperature. Then the catalyst was dried in an oven at 120° C. overnight and finally calcined at 300° C. for 2 hours in a muffle furnace.

### Example 2

### Preparation of Hydrogenation Catalyst Comprising 0.5% Pd Supported on 72 Weight % ZSM-5 and 18 Weight % Alumina

1.305 g of palladium nitrate salt was dissolved in 120 cc of water. The palladium solution was added to 120 g of the same alumina (20% alumina) bound ZSM-5 zeolite described in Example 1. The water was removed in a rotary evaporator by heating slowly to 65° C. The vacuum-dried material was dried in air in an oven at 120° C. overnight and finally calcined at 300° C. for 2 hours n a muffle furnace.

### Example 3

### Activation of Synthesis Gas Conversion Catalyst ex situ

Ten grams of catalyst as prepared in Example 1 was charged to a glass tube reactor. The reactor was placed in a muffle furnace with upward gas flow. The tube was purged first with nitrogen gas at ambient temperature, after which time the gas feed was changed to pure hydrogen with a flow rate of 750 sccm. The temperature of the reactor was increased to 350° C. at a rate of 1° C./minute and then held constant for six hours. After this time, the gas feed was switched to nitrogen to purge the system and the unit was then cooled to ambient temperature. Then a gas mixture of 1 volume % O₂/N₂ was passed up through the catalyst bed at 750 sccm for 10 hours to passivate the catalyst. No heating was applied, but the oxygen chemisorption and partial oxidation exotherm caused a momentary temperature rise. After 10 hours, the gas feed was changed to pure air, the flow rate was lowered to 200 sccm and the temperature was raised to 300° C. at a rate of 1° C./minute and then held constant for two hours. The catalyst was cooled to ambient temperature and discharged from the glass tube reactor.

### Example 4

### Stacked Bed Catalyst (Synthesis Gas Conversion and Hydrogenation Catalyst) Activation in situ

Ten grams of the catalyst from Example 3 diluted with 10 grams of gamma-alumina and the catalyst from Example 2 were transferred to a 316-SS tube reactor of 0.5″ inner diameter in series with the catalyst from Example 3 placed upstream of the catalyst from Example 2 and separated from it by a one gram layer of gamma-alumina. The reactor was then placed in a clam-shelf furnace. The catalyst beds were flushed with a downward flow of helium for a period of two hours, after which time the gas feed was switched to pure hydrogen at a flow rate of 500 sccm. The temperature was slowly raised to 120° C. at a temperature interval of 1° C./minute, held constant for a period of one hour, then raised to 250° C. at a temperature interval of 1° C./minute and held constant for 10 hours. After this time, the catalyst beds were cooled to 180° C. while remaining under a flow of pure hydrogen gas. All flows were directed downward.

### Comparative Example 1

### Synthesis Gas Conversion Using Catalyst of Example 1

A catalyst from Example 1 was activated as described in Example 3 and Example 4 and subjected to synthesis conditions in which 20 grams of the catalyst and support (10 g of catalyst and 10 g of alumina) was contacted with feed gas of hydrogen and carbon monoxide in ratios between 1.6 and 2.0 at temperatures between 205° C. and 225° C. with a total pressure of 10 atm and a total gas flow rate of 978 to 1951 cubic centimeters of gas per gram catalyst per hour. No downstream bed of Pd/ZSM-5 was present. The results are set forth in Table 1. At these conditions, there is a significant amount of solid wax formed without the aid of the downstream bed of Pd/ZSM-5.

TABLE 1

| | |
|---|---|
| Time on stream, hr | 21 |
| Temperature, ° C. | 220 |
| Pressure, atm | 10 |
| WHSV, mL/g/h | 2100 |
| H₂/CO nominal | 1.6 |
| H₂/CO usage | 2.14 |
| CO/(H₂ + N₂ + CO) | 0.35 |
| Recycle Ratio | 0 |
| % H₂ Converted | 69.67% |
| % CO Converted | 54.86% |
| Rate, gCH₂/g/h | 0.25 |
| Rate, mLC₅+/g/h | 0.16 |
| % CH₄ | 9.57% |
| % C₂ | 1.08% |
| % C₃ + % C₄ | 7.10% |
| % C₅+ | 82.24% |
| Wax | 8.00% |

### Example 5

### Preparation of Synthesis Gas Conversion Catalyst Comprising 20%, Cobalt-0.5% Ruthenium-1.0% Lanthanum Oxide Supported on Alumina

70 grams of extrudate of a gamma-alumina (Ketjen CK-300 commercially available from Akzo Chemie) which had been ground and sieved to 16-30 mesh size (0.589 mm-1.168 mm) and heated in air at 750° C. for 16 hours was used as a catalyst support. Separate portions comprising 0.1680 gram of ruthenium acetylacetonate, 2336 grams of lanthanum nitrate, and 87.563 grams of cobalt nitrate hexahydrate were dissolved in 181 cubic centimeters of acetone. The solution was divided into three equal parts and the alumina was contacted with the first portion of the catalyst solution with stirring. The solvent was removed from the impregnated alumina in a rotary evaporator at 40° C. The dried material was then calcined in air at 300° C. for two hours. The calcined catalyst was then impregnated with the second portion of the catalyst solution and the drying and calcining steps were repeated. The calcined catalyst was then impregnated, dried, and calcined as before for a third time. The catalyst analyzed 20.0 weight percent cobalt, 1.0 weight percent lanthanum oxide, 0.5 weight percent ruthenium, and the remainder alumina.

The catalyst was activated using the procedure outlined in Example 3.

### Example 6

### Synthesis Gas Conversion, Hydrocracking and Hydroisomerization Using Synthesis Gas Conversion Catalyst of Example 5 and Hydrogenation Catalyst of Example 2

Approximately 250 mg of synthesis gas conversion catalyst from Example 5 sized to 125-160 μm were diluted with 250 mg of SiC sized to 125-160 μm. Approximately 625 mg of hydrogenation catalyst from Example 2 was sized to 125-160 μm. A 5 mm inner diameter reactor tube was loaded in a “stacked bed” arrangement with the catalyst from Example 2 as the lower or downstream catalyst bed and the catalyst from Example 5 as the upper or upstream catalyst bed (“Catalyst 1” in Table 2). An identical reactor tube was loaded with only 250 mg of the catalyst from Example 5, sized to 125-160 μm and similarly diluted with 250 mg SiC (“Catalyst 2” in Table 2). The beds were activated in situ by the procedures outlined in Example 3 and Example 4.

The dual catalyst beds were subjected to synthesis conditions in which the catalyst was contacted with hydrogen and carbon monoxide at a ratio of 1.6-2.0 at temperatures between 205° C. and 210° C. with a total pressure of 10 atm and a total gas flow rate (weight hourly space velocity) of 8000 cubic centimeters of gas (0° C., 1 atm) per gram of Example 1 catalyst per hour using a high-throughput screening reactor as supplied by hte AG (Heidelberg, Germany). The total weight hourly space velocity was 2285 cubic centimeters of gas per gram of catalyst in both beds per hour. The process conditions and results are set forth in Table 2. The resulting liquid hydrocarbons were liquid at 0° C.

The degree of saturation of C₂-C₄ hydrocarbons, the amount of C₂₁+ product or Fischer-Tropsch wax, the degree of branching of C₄ hydrocarbons and the alpha number of the total product slate are all relative indicators of how effective the downstream Pd/ZSM-5 bed is at reducing, hydrocracking and hydroisomerizing the combined product resulting from the upstream catalyst bed. The results in Table 2 clearly show the efficacy of the Pd/ZSM-5 downstream bed for reducing, hydrocracking and hydroisomerizing activity. For example, while methane yield is similar for both catalyst systems, as expected, the percentage of C₁-C₄ is higher and the percentage of C₅+ is lower with the stacked bed dual catalyst system, indicative of hydrocracking activity of the lower catalyst bed of Pd/ZSM-5. In addition, the significantly higher ratio of paraffin/olefin for C₂-C₄ hydrocarbons using the dual bed catalyst system is evidence for the strong hydrogenation activity of the downstream Pd/ZSM-5 catalyst component. Furthermore, hydrocracking and hydroisomerization activity of the dual bed catalyst system is demonstrated by the much higher percentage of 2-butene isomers and the degree of C₄ branching.

A measure of the carbon number distribution is the Schulz-Flory alpha value, which represents the probability of making the next higher carbon number compound from a given carbon number compound. The Schulz-Flory distribution is expressed mathematically by the Schulz-Flory equation:

Wᵢ=(1−α)²iαⁱ⁻¹

where i represents carbon number, α is the Schulz-Flory distribution factor which represents the ratio of the rate of chain propagation to the rate of chain propagation plus the rate of chain termination, and Wᵢ represents the weight fraction of product of carbon number i. Alpha numbers above about 0.9 are, in general, representation of wax producing processes, and the higher the alpha number, e.g., as it approaches 1.0, the more selective the process is for producing wax molecules. Table 2 illustrates a considerable difference in alpha values between the two catalyst systems; the alpha value for the product arising from the dual-bed catalyst system containing the downstream Pd/ZSM-5 catalyst exhibits a far lower alpha value than does the product resulting from a conventional Fischer-Tropsch catalyst. This important difference is further highlighted by the very low percentage of Fischer-Tropsch wax in the dual bed catalyst system compared to that seen using a conventional Fischer-Tropsch catalyst.

TABLE 2

| | | | | | | |
|---|---|---|---|---|---|---|
| Time on stream, hr | 187 | 188 | 248 | 249 | 1089 | 1090 |
| Catalyst type | 1 | 2 | 1 | 2 | 1 | 2 |
| Temperature, ° C. | 205 | 205 | 205 | 205 | 210 | 210 |
| Pressure, bar | 10 | 10 | 10 | 10 | 10 | 10 |
| H₂/CO (inlet) | 2.0 | 2.0 | 1.6 | 1.6 | 2.0 | 2.0 |
| % CO Converted | 38 | 39 | 28 | 29 | 36 | 36 |
| % CH₄ | 8.9 | 8.7 | 7.4 | 7.2 | 9.6 | 9.4 |
| % C₂ | 1.5 | 1.0 | 1.4 | 0.8 | 1.5 | 1.0 |
| % C₃ + % C₄ | 15.1 | 8.5 | 14.0 | 7.7 | 11.1 | 8.4 |
| % C₅+ | 74.5 | 81.9 | 77.2 | 84.3 | 77.8 | 81.2 |
| Wax, % | <2 | 13 | <2 | 17 | <2 | 16 |
| % ethane | 100 | 92 | 100 | 87 | 100 | 93 |
| % ethene | 0 | 8 | 0 | 13 | 0 | 7 |
| % propane | 95 | 30 | 93 | 22 | 94 | 31 |
| % propene | 5 | 70 | 7 | 78 | 6 | 69 |
| % n-butane | 77.2 | 38.8 | 69.3 | 29.7 | 76.5 | 39.7 |
| % i-butane | 6.2 | 0.2 | 5.3 | 0 | 2.5 | 0 |
| % 1butene | 1 | 53.3 | 1.6 | 64.8 | 1.7 | 53.1 |
| % i-butene | 9.3 | 1.2 | 13.6 | 0 | 8.7 | 0 |
| % cis-2-butene | 2.5 | 4.0 | 4.0 | 2.8 | 4.1 | 3.7 |
| % trans-2-butene | 3.8 | 2.5 | 6.2 | 1.6 | 6.5 | 2.4 |
| % DOB C₄ | 15.5 | 1.4 | 18.9 | 0 | 11.2 | 0 |
| alpha 4-12 | 0.819 | 0.852 | 0.827 | 0.861 | 0.830 | 0.846 |

### Example 7

### Synthesis Gas Conversion, Hydrocracking and Hydroisomerization Using Synthesis Gas Conversion Catalyst of Example 1 and Hydrogenation Catalyst of Example 2

The single reactor containing the dual catalyst beds as described in Example 4 was subjected to synthesis conditions in which the catalyst was contacted with hydrogen and carbon monoxide at a ratio of 2.0 at temperatures between 220° C. and 225° C. with a total pressure of 10 atm and a total gas flow rate of 1900 cubic centimeters of gas (0° C., 1 atm) per gram of Example 1 catalyst per hour. Results are set forth in Table 3. The resulting liquid hydrocarbons were liquid at 0° C. Note that under the conditions of this experiment there is produced a high percentage of C5+ liquid product and no solid wax formation, illustrating the effectiveness of the downstream bed of Pd/ZSM-5 at the lower temperatures required for cobalt-catalyzed Fischer-Tropsch synthesis.

TABLE 3

| | | | | | | |
|---|---|---|---|---|---|---|
| Time on | 122 | 151 | 240 | 313 | 338 | 384 |
| stream, hr | | | | | | |
| Temperature, | 220 | 220 | 220 | 225 | 225 | 225 |
| ° C. | | | | | | |
| Pressure, atm | 10 | 10 | 10 | 10 | 10 | 10 |
| WHSV, | 1900 | 1900 | 1900 | 1900 | 1900 | 1900 |
| mL/g/h | | | | | | |
| H₂/CO inlet | 2.00 | 2.00 | 2.00 | 2.00 | 2.00 | 2.00 |
| H₂/CO usage | 2.36 | 2.37 | 2.39 | 2.26 | 2.20 | 2.30 |
| CO/(H₂ + | 0.33 | 0.33 | 0.33 | 0.33 | 0.33 | 0.33 |
| N₂ + CO) | | | | | | |
| Recycle Ratio | 0 | 0 | 0 | 0 | 1 | 0 |
| % H₂ | 72.9% | 68.3% | 63.0% | 87.9% | 80.4% | 81.1% |
| Converted | | | | | | |
| % CO | 61.9% | 57.5% | 52.9% | 77.7% | 73.1% | 70.6% |
| Converted | | | | | | |
| Rate, gCH₂/g/h | 0.24 | 0.23 | 0.21 | 0.30 | 0.29 | 0.28 |
| Rate, mLC₅+/ | 0.24 | 0.22 | 0.20 | 0.30 | 0.30 | 0.27 |
| g/h | | | | | | |
| % CH₄ | 14.6% | 14.1% | 14.9% | 15.5% | 12.9% | 15.6% |
| % C₂ | 1.7% | 1.6% | 1.7% | 1.7% | 1.5% | 1.7% |
| % C₃ + % C₄ | 10.3% | 10.1% | 11.0% | 9.1% | 7.0% | 9.7% |
| % C₅+ | 73.4% | 74.3% | 72.5% | 73.6% | 78.6% | 73.1% |
| Wax, g | 0 | 0 | 0 | 0 | 0 | 0 |

### Comparative Example 2

### Synthesis Gas Conversion Using 10% Cobalt Catalst Compared with a Stacked Bed of 10% Cobalt Synthesis Gas Conversion Catalyst and 0.5% Pd/ZSM-5 Hydrogenation Catalyst

A 5 mm inner diameter reactor tube was loaded with 500 mg of the catalyst from Example 1, sized to 125-160 μm (“Catalyst Type 3” in Table 4). An identical reactor tube was loaded in a stacked bed arrangement with 500 mg each of the catalyst from Example 2 as the lower or downstream catalyst bed and the catalyst from Example 1 as the upper or upstream catalyst bed (“Catalyst Type 4” in Table 4). The beds were activated in situ by the procedures described in Example 3 and Example 4.

The dual catalyst beds were subjected to synthesis conditions in which the catalyst was contacted with hydrogen and carbon monoxide at a ratio of 2.0 at 205° C. and a ratio of 1.5 at 215° C. and 225° C., with a total pressure of 10 atm and a total gas flow rate of 4000 cubic centimeters of gas (0° C., 1 atm) per gram of Example 1 catalyst per hour (weight hourly space velocity) using a high-throughput screening reactor as supplied by hte AG (Heidelberg, Germany). Based on the total weight of the dual beds, the weight hourly space velocity was 2000 cubic centimeter of gas per gram of catalyst per hour. The process conditions and results are set forth in Table 4. Flow rates in Table 4 are given as gas hourly space velocity (cubic centimeters of gas per cubic centimeter of catalyst per hour), based on the total weight for the dual catalyst beds.

It can be determined from a comparison of these results that the paraffin:olefin ratio, the alpha value and the degree of branching (DOB) of the C4-isomers all indicate that the downstream bed of Pd/ZSM-5 is effective at both hydroisomerization as well as hydrocracking even under the relatively mild conditions employed for the syngas conversion reaction.

TABLE 4

| | | | | | | |
|---|---|---|---|---|---|---|
| Time on stream, | 102 | 104 | 315 | 317 | 399 | 401 |
| hr | | | | | | |
| Catalyst type | 3 | 4 | 3 | 4 | 3 | 4 |
| Temperature, ° C. | 205 | 205 | 215 | 215 | 225 | 225 |
| Pressure, bar | 10 | 10 | 10 | 10 | 10 | 10 |
| H₂/CO usage | 2 | 2 | 1.5 | 1.5 | 1.5 | 1.5 |
| GHSV, hr⁻¹ | 4000 | 1500 | 4000 | 1500 | 4000 | 1500 |
| % CO | 23.3 | 23.3 | 26.2 | 27.4 | 40.9 | 41.8 |
| Converted | | | | | | |
| % C₁ | 13.6 | 13.2 | 11.5 | 11.5 | 12.5 | 12.4 |
| % C₁ + C₂ | 14.9 | 14.7 | 12.8 | 12.9 | 14.1 | 14.1 |
| % C₃ + C₄ | 11.0 | 16.1 | 10.4 | 15.3 | 11.7 | 14.7 |
| % C₅ − C₁₂ | 44.7 | 55.2 | 51.3 | 61.4 | 55.3 | 62.0 |
| % C₅+ | 75.7 | 72.1 | 78.4 | 75.3 | 76.3 | 74.6 |
| % C₁₃+ | 31.0 | 16.9 | 27.1 | 13.9 | 20.9 | 12.6 |
| % C₂₁+ | 8.3 | 2.3 | 5.6 | 1.7 | 4.9 | 1.9 |
| % Paraffin C₂ | 91.6 | 100.0 | 90.8 | 100.0 | 94.3 | 100.0 |
| % Olefin C₂ | 8.4 | 0 | 9.2 | 0 | 5.7 | 0 |
| % Paraffin C₃ | 29.9 | 93.6 | 27.7 | 85.9 | 37.7 | 87.1 |
| % Olefin C₃ | 70.2 | 6.4 | 72.3 | 14.2 | 62.3 | 12.9 |
| % Degree of | 4.3 | 25.2 | 5.8 | 34.4 | 8.6 | 36.3 |
| branching C₄ | | | | | | |
| % n-butane | 20.3 | 63.2 | 17.4 | 43.5 | 18.3 | 43.5 |
| % i-butane | 0.4 | 8.8 | 0.5 | 7.5 | 0.5 | 8.4 |
| % 1-butene | 13.2 | 1.6 | 13.4 | 3.1 | 12.3 | 3.0 |
| % i-butene | 3.9 | 16.5 | 5.3 | 26.9 | 8.1 | 27.9 |
| % cis-2-butene | 24.2 | 3.9 | 24.7 | 7.5 | 23.7 | 6.9 |
| % trans-2- | 38.0 | 6.1 | 38.6 | 11.5 | 37.1 | 10.3 |
| butene | | | | | | |
| alpha 4-12 | 0.841 | 0.781 | 0.867 | 0.781 | 0.840 | 0.783 |

### Comparative Example 3

### Comparison of Synthesis Gas Conversion Using Synthesis Gas Conversion Catalyst Alone, Stacked Bed Including H-ZSM-5 Hydrogenation Catalyst and Stacked Bed Including 0.5% Pd/ZSM-5 Hydrogenation Catalyst

Table 5 gives the process conditions and results for 250 mg of a 20% cobalt Fischer-Tropsch catalyst alone (Example 5 catalyst, referred to in the table as “Catalyst Type 5”), 250 mg of a 20% cobalt Fischer-Tropsch catalyst over 625 mg of H-ZSM-5 (weight ratio of 1:25, cobalt Fischer-Tropsch catalyst over H-ZSM-5) in a stacked bed arrangement (“Catalyst Type 6”), and 250 mg of a 20% cobalt Fischer-Tropsch catalyst over 625 mg of 0.5% Pd/ZSM-5 (having a weight ratio of 1:2.5) in a stacked bed arrangement (“Catalyst Type 7”).

A comparison of the results in Table 5 shows that while the dual bed with the H-ZSM-5 component (Catalyst Type 6) shows some cracking activity compared to the 20% cobalt Fischer-Tropsch catalyst alone (Catalyst Type 5), the presence of the palladium Group VIII metal serves to dramatically enhance both the hydroisomerization and hydrocracking activity of the ZSM-5 component.

TABLE 5

| | | | | | | | | | |
|---|---|---|---|---|---|---|---|---|---|
| Time on | 109 | 104 | 108 | 518 | 317 | 517 | 828 | 401 | 835 |
| stream, hr | | | | | | | | | |
| Catalyst type | 5 | 6 | 7 | 5 | 6 | 7 | 5 | 6 | 7 |
| Temperature, | 205 | 205 | 205 | 215 | 215 | 215 | 225 | 225 | 225 |
| ° C. | | | | | | | | | |
| Pressure, bar | 10 | 10 | 10 | 10 | 10 | 10 | 10 | 10 | 10 |
| H₂/CO usage | 2 | 2 | 2 | 1.5 | 1.5 | 1.5 | 1.5 | 1.5 | 1.5 |
| GHSV, hr⁻¹ | 7000 | 1500 | 1200 | 7000 | 1500 | 1200 | 7000 | 1500 | 1200 |
| % CO | 42 | 39 | 41 | 40 | 48 | 41 | 55 | 59 | 54 |
| Converted | | | | | | | | | |
| % C₁ | 8.1 | 11.3 | 8.3 | 8.1 | 10.6 | 7.1 | 8.6 | 14.9 | 8.1 |
| % C₁ + C₂ | 9.0 | 12.7 | 9.8 | 9.1 | 12.2 | 8.4 | 9.8 | 17.4 | 9.7 |
| % C₃ + C₄ | 7.9 | 10.1 | 17.1 | 7.5 | 9.9 | 11.0 | 8.0 | 12.2 | 13.4 |
| % C₅ − C₁₂ | 42.3 | 40.5 | 66.5 | 42.1 | 42.9 | 59.9 | 36.4 | 48.1 | 58.7 |
| % C₅+ | 83 | 79 | 76 | 83 | 79 | 82 | 79 | 72 | 79 |
| % C₁₃+ | 40.8 | 38.1 | 9.6 | 41.2 | 36.4 | 22.1 | 47.0 | 24.3 | 20.6 |
| % Wax | 17 | >9 | <2 | 15 | >7 | <2 | 15 | >7 | <2 |
| % Degree of | 1.4 | 4.8 | 18.6 | 1.7 | 5.8 | 6.8 | 2.3 | 8.7 | 23.7 |
| branching C₄ | | | | | | | | | |
| % n-butane | 39.9 | 43.9 | 75.6 | 34.8 | 41.1 | 78.9 | 41.7 | 44.6 | 65.0 |
| % i-butane | 0.2 | 0.5 | 9.6 | 0.2 | 0.5 | 6.8 | 0.3 | 0.7 | 7.5 |
| % 1butene | 51.6 | 6.9 | 0.8 | 54.9 | 7.6 | 2.0 | 44.6 | 6.9 | 1.7 |
| % i-butene | 1.2 | 4.3 | 9.0 | 1.5 | 5.3 | 0.0 | 2.0 | 7.9 | 16.2 |
| % cis-2-butene | 4.4 | 16.6 | 2.0 | 4.6 | 17.2 | 4.9 | 6.7 | 15.3 | 3.9 |
| % trans-2- | 2.8 | 27.8 | 3.1 | 3.0 | 28.4 | 7.4 | 4.8 | 24.6 | 5.8 |
| butene | | | | | | | | | |
| alpha 4-12 | 0.87 | 0.8 | 0.8 | 0.85 | 0.8 | 0.8 | 0.85 | 0.8 | 0.8 |

### Example 8

### Cloud Point, Freeze Point and Pour Point Analysis Using Synthesis Gas Conversion Catalyst of Example 1 and Hydrogenation Catalyst of Example 2

The single reactor containing the dual catalyst beds as described in Example 4 was subjected to synthesis conditions in which the catalyst was contacted with hydrogen and carbon monoxide at a ratio of 1.6, a temperature of 220° C. and a total pressure of 10 atm.

The cloud point of the product sample was determined to be approximately 6° C. Cloud point refers to the temperature below which wax in a liquid hydrocarbon product forms a cloudy appearance. The presence of solidified waxes in conventional fuels thickens the product and clogs fuel filters and injectors in engines. The wax also accumulates on cold surfaces and forms an emulsion with water. Therefore, cloud point indicates the tendency of the product to plug filters or small orifices at cold operating temperatures. Note that a 6° C. cloud point is typical for a Number 2 diesel.

The freeze point of the product sample was determined to be approximately −6.4° C. Freeze point (also referred to as gel point) refers to the temperature below which solid wax particles are large enough to be stopped by a fuel filter.

The pour point of the product sample was determined to be less than −60° C., or below the lower limit of the measuring device used, indicating that the product can easily be transported at low temperatures. Pour point is a practical measure of the ease of pouring and pumping a liquid hydrocarbon product. Pour point temperature is determined as follows. A product sample in a jar is cooled inside a cooling bath to allow the formation of paraffin wax crystals. At about 9° C. above the expected pour point, and for every subsequent 3° C., the jar is removed and tilted to check for surface movement. When the sample does not flow when tilted, the jar is held horizontally for five seconds. If the product sample ceases to flow, 3° C. is added to the corresponding temperature and the result is the pour point temperature.

While various embodiments have been described, it is to be understood that variations and modifications may be resorted to as wilt be apparent to those skilled in the art. Such variations and modifications are to be considered within the purview and scope of the claims appended hereto.

What is claimed is:

1. A process for converting synthesis gas to liquid hydrocarbons in a single reactor comprising:
contacting a feed comprising a mixture of carbon monoxide and hydrogen with a first catalyst bed comprising a synthesis gas conversion catalyst and a second catalyst bed comprising a mixture of a hydrogenation catalyst and a solid acid catalyst downstream of the first catalyst bed at essentially common reaction conditions, such that a Fischer-Tropsch wax is formed over the first catalyst bed and said wax is hydrocracked and hydroisomerized over the second catalyst bed, thereby resulting in liquid hydrocarbons substantially free of solid wax.

2. The process of claim 1 wherein the synthesis gas conversion catalyst comprises cobalt on a solid oxide support.

3. The process of claim 2 wherein the solid oxide support is selected from the group consisting of alumina, silica, and titania.

4. The process of claim 1 wherein the synthesis gas conversion catalyst comprises cobalt supported on an acidic component.

5. The process of claim 1 wherein the hydrogenation catalyst comprises a Group VIII metal selected from the group consisting of rhodium, iridium, palladium and platinum.

6. The process of claim 1 wherein the solid acid catalyst comprises a zeolite.

7. The process of claim 6 wherein the zeolite is a medium pore molecular sieve.

8. The process of claim 1 wherein the hydrogenation catalyst is directly supported on the solid acid catalyst.

9. The process of claim 1 wherein the hydrogenation catalyst and the solid acid catalyst are intimately mixed.

10. The process of claim 1 wherein the reactor temperature is between about 160° C. and about 260° C.

11. The process of claim 1 wherein the reactor temperature is between about 175° C. and about 250° C.

12. The process of claim 1 wherein the reactor temperature is between about 185° C. and about 235° C.

13. The process of claim 1 wherein the temperature of the first catalyst bed and the temperature of the second catalyst bed differ by no more than about 20° C.

14. The process of claim 1 wherein the synthesis gas conversion catalyst further comprises a promoter selected from the group consisting of ruthenium, rhenium, platinum, palladium, gold, and silver.

15. The process of claim 1 wherein the liquid hydrocarbons produced comprise:
0-20 weight % CH₄;
0-20 weight % C₂-C₄;
50-95 weight % C₅₊; and
0-8 weight % C₂₁₊.

16. The process of claim 1 wherein the gaseous hourly space velocity is less than about 20,000 volumes of gas per volume of catalyst per hour.

17. The process of claim 1 wherein the gaseous hourly space velocity is between about 100 and about 5000 volumes of gas per volume of catalyst per hour.

18. The process of claim 1 wherein the gaseous hourly space velocity is between about 1000 and about 2500 volumes of gas per volume of catalyst per hour.

19. The process of claim 1 wherein the reaction pressure is between about 1 atmospheres and about 100 atmospheres.

20. The process of claim 1 wherein the reaction pressure is between about 3 atmospheres and about 35 atmospheres.

21. The process of claim 1 wherein the reaction pressure is between about 5 atmospheres and about 20 atmospheres.
